(19) United States
(12) Patent Application Publication
(10) Pub. No.: US 2022/0288523 A1
(43) Pub. Date: Sep. 15, 2022

(54) SYSTEM AND METHOD FOR THE IDENTIFICATION AND SEPARATION OF COMPOUNDS CARRIED IN A FLUID STREAM

(71) Applicant: Phuc Labs, Inc., Boston, MA (US)

(72) Inventors: Phuc Vinh Truong, Boston, MA (US); Midnight Zero, Wilmington, MA (US); Phuc Thien Truong, Boston, MA (US)

(21) Appl. No.: 17/682,895

(22) Filed: Feb. 28, 2022

Related U.S. Application Data
(60) Provisional application No. 63/155,162, filed on Mar. 1, 2021.

(51) Int. Cl. B01D 46/44

(52) U.S. Cl. CPC B01D 46/442; B01D 2201/54; B01D 2247/08; B01D 2273/26

(57) ABSTRACT

Described herein is a method of identifying components in a fluid stream. Particularly, the invention relates to the identification and/or separation of one or more materials suspended in a fluid stream.

### RELATED APPLICATIONS

This application claims the benefit of U.S. Provisional Application No. 63/155,162, filed on Mar. 1, 2021. The content of which is incorporated herein by reference in its entirety.

### BACKGROUND OF THE INVENTION

### Field of the Invention

The invention generally relates to the identification of compounds carried in a fluid stream. Particularly the invention relates to the identification and separation of one or more compounds carried in a fluid stream.

### Background

Many chemical processes create mixtures that require separation of the various chemical compounds of the mixture. A variety of techniques exist to effect such separations. In the case when the mixture is a liquid or gas fluid that includes one more suspended compounds, the suspended compounds are removed from the fluid mixture to provide a relatively clean fluid stream. A used herein a fluid stream that includes suspended compounds can be a mixture of two different liquid fluids (e.g., oil (hydrocarbons) and water) or a solid and a fluid (e.g., solid metal particles suspended in a liquid or smoke particles suspended in air).

Techniques for separating compounds in a fluid steam typically use filter systems. Exemplary filter systems include screens, porous membranes, and porous media filters which may have chemical treatments to enhance or increase specificity of separation. Another type of filter is electrostatic particle separation, magnetic separation, etc. These types of filter lack specificity beyond simple attributes such as size or chemical reactivity and typically require significant size and/or pressure drop compared to the unfiltered flow of the fluid stream, as well as replacement and/or cleaning.

Suspended compounds in a fluid stream may separate from each other if the fluid is not disturbed over time. The less-dense compound will rise to the top of the container and the more-dense compound will sink to the bottom of the container. When the fluid is a liquid, the isolation of each layer can be accomplished by sequential removal of each layer from a container. For example, water and oil will separate into layers if left undisturbed in a storage container. Once the fluid layers are separated, each layer can be removed from the container. This type of separation is complicated by the need to identify when the first layer (e.g., the dense layer) has been removed so that the receiving container can be switched to receive the second layer (e.g., the less dense layer). This is further complicated when mixtures of unknown quantities of each compound need to be separated.

In other processes, rather than separating the compounds in a fluid stream, it may be desirable to simply determine the amount of compounds that are carried in a fluid stream. For example, it may be desirable to determine the amount of specific particulate solids that are suspended in a liquid or gaseous fluid stream or the general amount of materials in a fluid stream. Typically, this can be accomplished by taking a crude measurement of the concentration of each compound, without the use of any separating after measurement.

These types of fluid processing processes could benefit from an improved method of identifying compounds carried in a fluid stream, particularly while the fluid stream is in motion.

### SUMMARY OF THE INVENTION

In an embodiment, a method of identifying and separating compounds carried in a fluid stream includes: passing the fluid stream through a detection system; determining the presence of compounds in the fluid stream as the fluid stream passes through the detection system; selecting a flow channel for the fluid stream to be sent based on one or more properties of the compounds in the fluid stream; and directing the fluid stream to the selected flow channel to separate the compounds.

In an embodiment, the fluid stream is a liquid fluid stream and the liquid fluid stream comprises an immiscible fluid suspended in a liquid fluid. In an embodiment, the fluid stream is a liquid fluid stream and the liquid fluid stream comprises one or more solid compounds carried in the liquid fluid. In an embodiment, the fluid stream is a gaseous fluid and the gaseous fluid stream comprises one or more solid compounds carried in the gaseous fluid.

In an embodiment, determining the properties of the compounds comprises transmitting electromagnetic radiation into the fluid stream passing through the detection system and detecting a signal response from the electromagnetic radiation. The electromagnetic radiation can be visible light, ultraviolet light, infrared light, or combinations thereof.

In an embodiment, the detection system includes a light source. The light source can be a visible light source, an ultraviolet light source, and an infrared light source, or combinations thereof. The light source may use a combination of ultraviolet, visible, or near infrared light to image objects (e.g., using a LIDAR system).

In an embodiment, determining the properties of the compounds comprises identifying the chemical composition of the compounds based on the interaction of the compounds with the electromagnetic radiation and selecting a flow channel for the fluid stream to be sent after leaving the detection system is based on the chemical composition of the compounds in the fluid stream.

In an embodiment, the method further comprises determining the concentration of the compounds in the fluid stream as the fluid stream passes through the detection system and selecting a flow channel for the fluid stream to be sent after leaving the detection system is based on the concentration of the compounds in the fluid stream.

In an embodiment, the detection system determines the concentration of the compounds carried in the fluid. In an embodiment, the detection system identifies the chemical composition of the compounds based on the interaction of the compounds with the light from a light source. In an embodiment, the detection system determines the shape of the compounds. In an embodiment, the detection system determines the light absorbance spectrum of the compounds. In an embodiment, the detection system determines the size of the compounds.

In an embodiment, a system for identifying and separating compounds carried in a fluid stream comprises: a detection system configured to receive the fluid stream. The detection system is configured to determine the properties of the compounds in the fluid stream as the fluid stream passes through the detection system. The system further includes a data analysis device coupled to the detector, wherein the data analysis device comprises a processor and a memory source, the processor operable to execute program instructions, and wherein the program instructions are operable to determine the presence of the compounds in the fluid stream as the fluid stream passes through the detection system. The system further includes one or more valves coupled to the detection system and the data analysis device. The system further includes a plurality of flow channels coupled to the one or more valves. During use, the data analysis device selects a flow channel for the fluid stream to be sent after leaving the detection system based on one or more properties of the compounds in the fluid stream. The data analysis device operates the one or more valves to direct the fluid stream to the selected flow channel after the fluid stream passes through the detection system to separate the material.

In an embodiment, a detection system for identifying compounds carried in a fluid stream comprises: a scanning area configured to receive the fluid stream; a light source optically coupled to the scanning area; a detector optically coupled to the scanning area, wherein the detector is positioned to receive light from the light source after the light passes through the scanning area; and a data analysis device coupled to the detector, wherein the data analysis device comprises a processor and a memory source, the processor operable to execute program instructions, and wherein the program instructions are operable to determine the presence and/or location of the compounds in the fluid stream as the fluid stream passes through the scanning area.

In an embodiment, the scanning area is composed of a material that is transmissive to visible light, infrared light, ultraviolet light, or a combination thereof.

In an embodiment, the system may include a pump. A storage container may be used to hold the compounds carried in the fluid stream before separation. In an embodiment, the pump receives the mixture of compounds in a fluid from the storage container and creates the fluid stream.

In an embodiment, the system also includes one or more receiving containers for receiving the fluid stream after the fluid stream passes through the scanning area.

In an embodiment, a method of identifying and separating compounds carried in a fluid stream comprises: passing the fluid stream through a detection system comprising a light source; passing light from a light source through a scanning area to a detector in the detection system while the fluid stream is passed through the scanning area; determining the concentration of the compounds carried in the fluid stream as the fluid stream passes through the scanning area; selecting a receiving container to collect the fluid stream passing through the scanning area based on the concentration of suspended materials in the fluid stream; and directing the fluid stream to the selected receiving container after the fluid stream passes through the scanning area.

In an embodiment, a method of separating a liquid hydrocarbon from a water stream, wherein the liquid hydrocarbon is immiscible in the water stream, the method comprises: collecting a mixture of water and the liquid hydrocarbon in a storage container and allowing the mixture to separate into a water layer and a hydrocarbon layer. The method further includes:
(a) removing the mixture from the storage tank;
(b) passing the removed mixture through a scanning area as a fluid stream;
(c) passing light from a light source through the scanning area to a detector while the fluid is passed through the scanning area;
(d) determining the concentration of the oil and/or water in the fluid stream as the fluid stream passes through the scanning area;
(e) selecting a receiving tank to collect the fluid passing through the scanning area based on the concentration of oil and/or water in the fluid stream; and
(f) directing the fluid stream to the selected receiving container after the fluid stream passes through the scanning area.

In an embodiment, a method of identifying and separating metal containing particles carried in a fluid stream comprises: passing the fluid stream through a detection system; identifying properties of the metal containing particles in the fluid stream as the fluid stream passes through the detection system; selecting a flow channel for the fluid stream to be sent after leaving the detection system based on the properties of the metal containing particles in the fluid stream; directing the fluid stream to the selected flow channel after the fluid stream passes through the detection system to separate the metal containing particles.

The properties of the metal containing particles that can be used to determine a flow channel for the metal containing particles include, but are not limited to, the chemical identity of the metal containing particles, the shape of the metal containing particles, and the size of the metal containing particles. The metal containing particles may be substantially pure metal particles or may be a metal containing mineral particle.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a schematic diagram of a system for identifying and/or separating suspended materials from a liquid fluid stream;

FIGS. 2A-C depicts different views of an exemplary system for identifying and/or separating suspended materials from a liquid fluid stream;

FIG. 3 depicts a cut-away side view of a detection system;

FIG. 4 depicts a projection, see-through view of a stream splitter;

FIG. 5 is a block diagram illustrating an example data analysis device, in accordance with one or more embodiments of the present disclosure;

FIG. 6 is a diagram illustrating an example neural network, in accordance with one or more embodiments of the present disclosure;

FIG. 7 is a flow diagram illustrating an example process for identifying/detecting suspended material and/or separating the suspended material from a fluid stream, in accordance with one or more embodiments of the present disclosure; and

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a,” “an,” and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

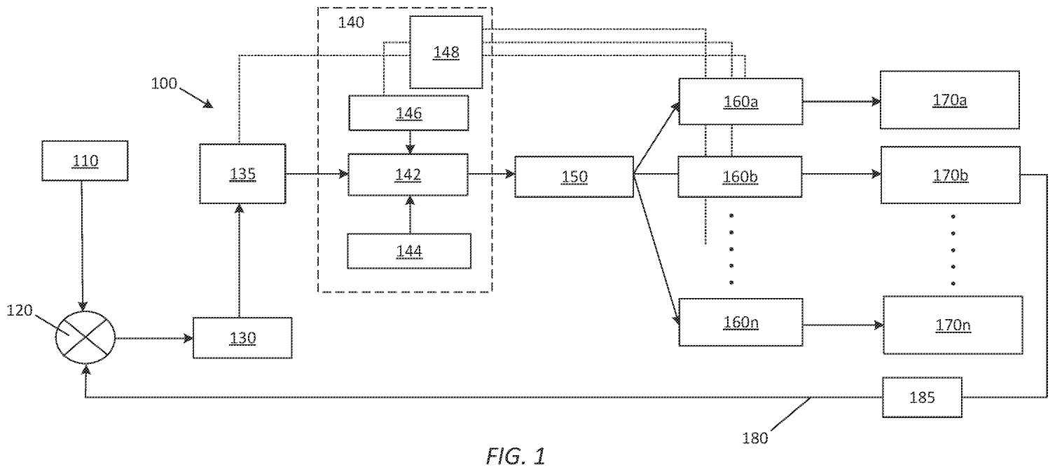

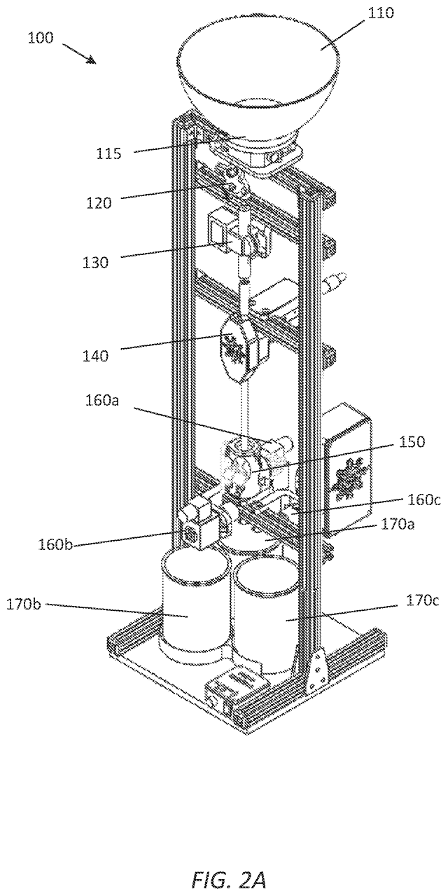

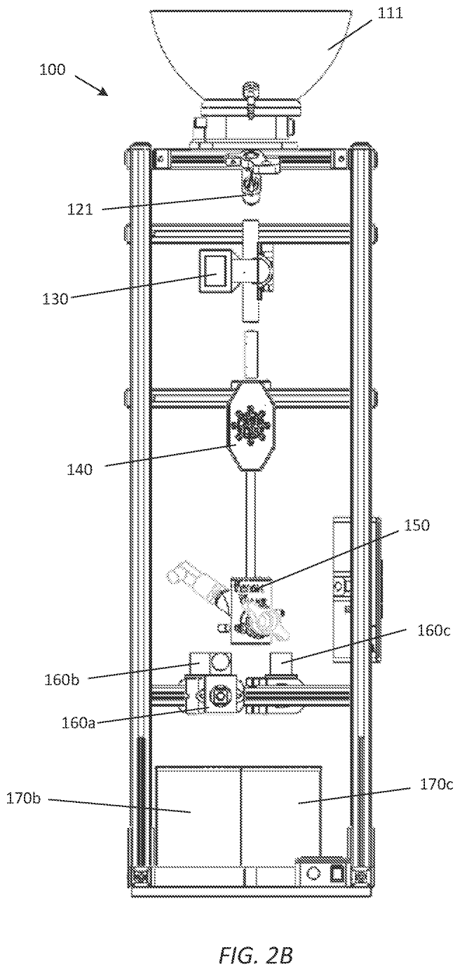

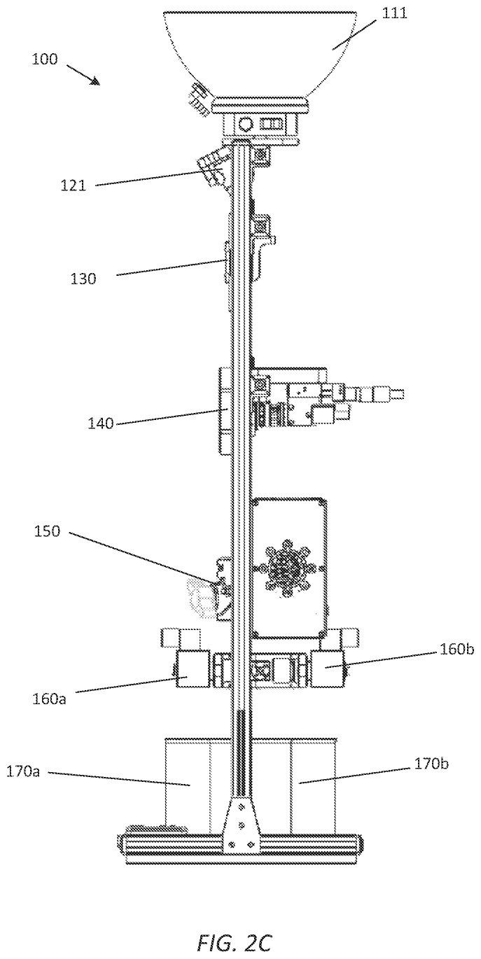

FIG. 1 depicts a system 100 that can be used to identify and separate compounds carried in a fluid stream. FIGS. 2A-2C depict an embodiment of an exemplary identification/separation system 100. In one embodiment, the fluid stream is a liquid fluid stream having an immiscible fluid compound carried in the liquid fluid stream (a “liquid-liquid mixture”). An example of a fluid having an immiscible fluid suspended therein, is a mixture of water and an immiscible liquid hydrocarbon. A hydrocarbon, as used herein, is any compound that is composed of, primarily, hydrogen and carbon. An immiscible hydrocarbon is a hydrocarbon that has a solubility on the liquid fluid of less than 0.1% by volume. If water is the fluid stream, immiscible hydrocarbons can be pure hydrocarbons such as pentane, hexane, heptane, octane, etc. Immiscible hydrocarbon liquids can also be mixtures of different hydrocarbon molecules. For example, vegetable oils are mixtures of saturated fatty acids (C8:0 to C18:0), monounsaturated fatty acids (C16:1, C18:1), and polyunsaturated fatty acids (C18:2 and C18:3). Immiscible hydrocarbon liquids can also include petroleum products such as crude oil and refined oil products.

In another embodiment, the fluid is a liquid fluid stream and the material is one or more solid compounds carried in the liquid fluid stream (a “solid-liquid mixture”). Examples of solid compounds that can be carried in a fluid stream include, but are not limited to, minerals, sand, catalysts, chemical coagulants, dirt and metal particles. Particles carried in a fluid stream can be any size and shape depending on the type of solid material and the source of the material. Solid particles can range from millimeter size down to micron size or nanometer size. As used herein, millimeter size particles have an average diameter of less than 25 mm. As used herein, micron-size particles have an average diameter of less than 1 mm. Nanoparticles are particles having an average diameter of less than 1 micron.

In another embodiment, the fluid is a gaseous fluid and the material is a solid suspended in the gaseous fluid (a “solid-gas mixture”). Smoke is an example of a gaseous fluid having a solid suspended therein. Clouds are examples of a solid or liquid or mix of solid and liquid materials suspended in a gas. Suspended materials in a gaseous material may be quite large such as millimeter size or larger which can be fluidized or transported along a surface by a gas for transport and/or separating

The fluid that is being analyzed and/or separated is stored in storage container 110. Storage container 110 serves as a collection source of the fluid to be analyzed. When a mixture is collected in the storage container, the mixture may separate into distinct layers. If the fluid is a liquid-liquid mixture, the mixture may separate into two or more layers based on the density of the components in the mixture. In a two-compound system, the compounds of the mixture with the highest density will settle below the compounds of the mixture having the lowest density. If more than one compound is in the fluid, the compounds will separate into multiple layers ordered from bottom to top based on the density of the compounds. If the fluid is a liquid-solid mixture, the mixture may separate into a solids layer and a liquid layer. The solids layer is typically on the bottom of the storage container due to the difference in density between the solid and the liquid. It is possible, however, that the solid layer may float on the liquid layer if the solid has a density that is less than the density of the liquid. If a solid-gas mixture is being analyzed, then a storage container may not be used, and the ambient environment may be used as the input fluid for the system. Additionally, it may be the case that the various compounds of the mixture remain mixed or may not separate before entering the system.

A fluid stream is created from the fluid stored in storage container 110. In an embodiment, fluid pump 120 is coupled to storage container 110. In an embodiment, an outlet conduit is positioned at or near the bottom of storage container 110. During use, fluid can be taken out of the storage container through the outlet conduit. The outlet conduit is coupled to a fluid pump 120. Fluid pump 120 is a suitable fluid pump for the fluid being studied. For example, the type of pump being used for a liquid-liquid mixture is different than a pump used for a liquid-solid mixture. Also, a different pump may be used for pumping a gaseous fluid. In one embodiment, a pump was used for creating a fluid stream of a liquid-liquid mixture. In an alternate embodiment, the system may rely on gravity flow for providing a fluid stream in which case the flow may be adjusted by adjustment of the heights of the free surfaces in the system and/or the angle of components of the system relative to gravity.

In the embodiment depicted in FIGS. 2A-C, the storage container may be a mixer 111. Mixer 111 may agitate or stir the fluid introduced into the mixer to create a suspension of materials that are substantially homogeneously suspended in the fluid. Mixer 111 may be a paddle or blade mixer, or a magnetic mixer. Mixer 111 may also agitate the fluid using ultrasound or vibrations. Mixer 111 may also be a vortex separator/mixer. After leaving mixer 111, the fluid stream may be introduced into the separation system through valve 121. Valve 121 may be any kind of valve such as a gate valve, ball valve, plug valve, butterfly valve, needle valve, etc.

The fluid stream generated by fluid pump 120 (or generated through release of fluid through valve 121) is sent to flow regulator 130. Flow regulator 130 is used to alter the flow rate of the fluid stream. Flow regulator 130 maintains a predefined flow rate independent of the pressure of the fluid stream generated by the fluid pump. Flow regulator 130 may be a constrictive flow regulator. A constrictive flow regulator has a central internal diameter that is smaller than the inlet diameter of the flow restrictor. The reduced diameter can accelerate the flow of water to a predetermined and constant rate as long as the inlet pressure is above a predefined minimum. A reduced diameter can also reduce the velocity or flow rate through the system. The constricted central diameter may be created by the physical properties of the flow regulator or by adjusting the internal diameter. Adjustable diameter flow regulators may be manually adjusted or remotely adjusted through a controller.

After passing through flow regulator 130, the fluid stream is conducted to flow meter 135. Flow meter 135 measures the speed of the fluid flowing through the flow meter. In one embodiment, flow meter 135 is electrically coupled to data analysis device 148, which continually or periodically monitors the flow rate. In some embodiments, as discussed later, it is desirable to ensure that the flow rate of the fluid stream is constant and within a predefined range of a predefined speed. In an exemplary embodiment, an Omega Low Flow Paddlewheel Flow Meter—FTB300 Series (Omega, Norwalk, Conn.) may be used to measure flow rates. It should be understood that the use of a flow meter is optional. If the fluid stream is not flowing at the proper speed, the speed of pump 120 may be altered to change the speed of the fluid stream. Alternatively, it may be necessary to change the flow regulator 130 or adjust the internal diameter of the flow regulator to alter the speed of the fluid stream.

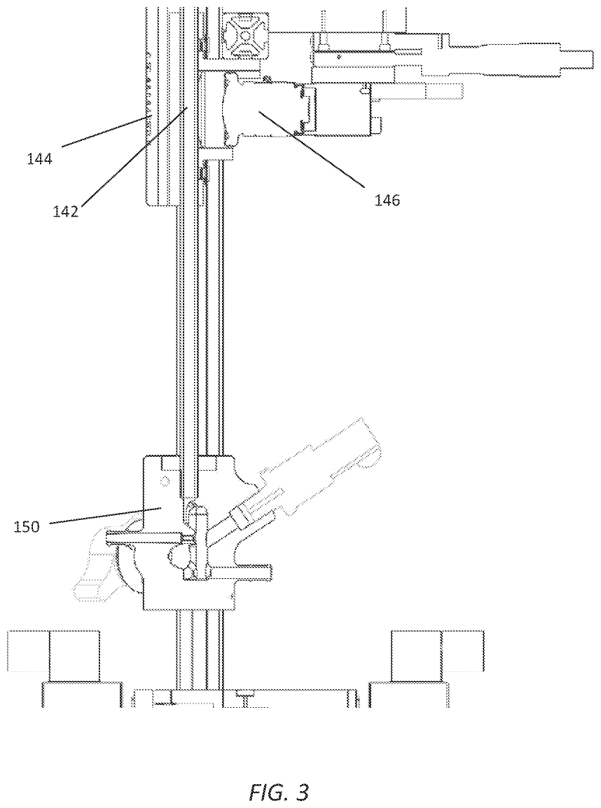

After passing through flow regulator 130, the fluid stream is passed into a detection system 140. Detection system 140 includes a scanning area 142, an electromagnetic radiation source 144, an electromagnetic radiation detector 146, and a data analysis device 148. In an embodiment, a camera may be used as a light detector (e.g., Edmund Optics BFS-U3-04S2c-CS USB 3.1 Blackfly® S Color Camera (Edmund Optics, Barrington, N.J.). The electromagnetic radiation source may be an LED device (e.g., LED light #1528-1710-ND (Digi-Key Electronics, Thief River Falls, Minn.). FIG. 3 shows a cross-sectional view of an embodiment of a detection system.

As shown in FIGS. 1 and 3, the fluid stream passes through scanning area 142. Electromagnetic radiation source 144 irradiates the scanning area 142 with electromagnetic radiation at a wavelength suitable for analysis of the compounds in the fluid stream. Radiation from electromagnetic radiation source 144 passes through the scanning area 142 as the fluid stream passes through the scanning area 142. Electromagnetic radiation is collected by the detector, after the fluid stream is irradiated with the electromagnetic radiation. The collected electromagnetic radiation is converted into information that is passed on to data analysis device 148, where the information is analyzed to determine the properties of the compounds in the fluid stream as the fluid stream passes through the scanning area 142.

The electromagnetic radiation interacts with the compounds in the fluid stream and this interaction is captured by the detector. In one embodiment, electromagnetic radiation can be transmitted through the fluid stream and compounds in the fluid stream absorb a portion of the electromagnetic radiation. The compounds can then be identified by analyzing the wavelengths of electromagnetic light that is absorbed by the compounds in the fluid stream. In another embodiment, the electromagnetic radiation can be transmitted through the fluid stream and compounds in the fluid stream reflect a portion of the electromagnetic radiation. The compounds can then be identified by analyzing the wavelengths of electromagnetic light that is reflected by the compounds in the fluid stream. In another embodiment, the electromagnetic radiation can be transmitted through the fluid stream and compounds in the fluid stream absorb a portion of the electromagnetic radiation. The compounds can then be identified by analyzing the wavelengths of electromagnetic radiation that is emitted by the compounds in the fluid stream.

Scanning area 142 is at least partially transmissive to at least a portion of the radiation produced by electromagnetic radiation source 144. In one embodiment, the entire scanning area 142 is formed from a material that is transmissive of at least a portion of the electromagnetic radiation produced by the electromagnetic radiation source. For example, the scanning area 142 may be optically transmissive for visible light, ultraviolet light, infrared light, or any combination of these lights. Suitable materials for the formation of the scanning area 142 include, but are not limited to, polycarbonate, borosilicate glass, and quartz glass. The scanning area can be an open flow, channel flow, or waterfall flow design.

In another embodiment, scanning area 142 may be formed from two materials, an opaque material and a radiation transmissive material(s) positioned to form a window through the opaque material. For example, windows formed from a light transmissive material may be oriented on opposing sides of an opaque scanning area 142 to allow light to pass from the electromagnetic radiation source, through the scanning area 142, onto the detector.

Electromagnetic radiation source 144 may be any electromagnetic radiation source that is suitable for the detection of the compounds in the fluid stream. For example, the electromagnetic radiation source may include a visible light source, an ultraviolet light source, an infrared light source, or any combination of these light sources. Suitable light sources include incandescent light sources, fluorescent light sources, halogen light sources, and light-emitting diode (LED) light sources. A light source may be a singular light source or may include a plurality of light sources. The light source may use a combination of ultraviolet, visible, or near infrared light to image objects (e.g., using a LIDAR system). Electromagnetic radiation source 144 may be positioned toward the radiation transmissive material of the scanning area 142 and opposite to detector 146.

Other forms of energy may be directed/passed through the scanning area. For example, electromagnetic radiation (e.g., radio waves, microwaves, X-rays, gamma rays, etc.) may be directed/passed through the scanning area by an energy source (e.g., an emitter or transmitter). In another example, ultrasound waves may be directed/passed through the scanning area. It is also possible that the components in the flow do not need excitation to be sensed. For example, if the compounds are naturally radioactive, only a radiation detector may be needed to identify the compounds.

Detector 146 may be any type of detector that is suitable for the detection of the wavelengths of radiation being absorbed or produced by the compounds in the fluid stream. For example, if the electromagnetic radiation source includes a visible light source, detector 146 is capable of capturing visible light. Detector 146 may also, or alternatively, be capable of detecting ultraviolet light and/or infrared light if needed for identification of the components of the fluid stream. Suitable detectors include but are not limited to CCD or CMOS cameras. A detector that includes a camera can be used to identify physical properties of the compounds such as size and shape of the compounds. Detector 146 may be positioned toward the optically transmissive material of the scanning area 142 and opposite to light source 144.

In some embodiments, other devices, components, systems, etc., may be used in the system 100 to enable analysis of the fluid stream. For example, the electromagnetic radiation source 144 may be replaced with any device, component, or system that may apply, generate, direct, emit, transmit, etc., a form of energy to the scanning area 142 and/or fluid stream. For example, the electromagnetic radiation source 144 may be any energy generation device or an energy source that can generate X-rays, microwaves, ultraviolet rays, ultrasound waves, etc. The detector 146 may be any device, component, or system that can detect the energy generated by the energy generation devices. For example, the detector 146 can include a receiver that may detect X-rays, microwaves, radiation, ultraviolet rays, ultrasound waves, etc.

In a specific embodiment, the fluid stream passes through scanning area 142, where the fluid stream is analyzed. The fluid stream may be analyzed to determine the compounds carried by the fluid stream as it passes through the scanning area 142. As the fluid stream passes through the detection system, the fluid stream is irradiated with radiation from electromagnetic radiation source 144. Electromagnetic radiation from the electromagnetic radiation source passes through the scanning area 142 and is collected in detection system 146. Information collected by the detection system is passed to the data analysis device 148 for determination of the properties of the compounds carried by the fluid stream through the detector. Properties of the compounds include, but are not limited to quantitative and qualitative properties of the compounds. Quantitative properties of the compounds include properties such as the amount of compounds or the concentration of the compounds passing through the detection system. For example, data analysis device may determine the concentration of the liquid droplets or solid particles carried by the fluid stream. The data analysis device may also, or alternatively, identify qualitative properties of the compounds carried in the fluid stream. Qualitative properties of the compounds include, the chemical composition of the compounds, the shape of the compounds, the size of the compounds, the absorbance of electromagnetic radiation by the compounds, and the emission of electromagnetic radiation in response to being subjected to electromagnetic radiation. The identification of the chemical composition of the compounds carried in the fluid stream can be based on the interaction of the material with the electromagnetic radiation from the electromagnetic radiation source. The data analysis device may also, or alternatively, identify the shape and/or size of compounds carried in the fluid stream based on the interaction of these materials with electromagnetic radiation (e.g., light) from the electromagnetic radiation source, using a camera in the detection system.

In one embodiment, the scanning area 142 may include a passageway through which the fluid stream may pass. The passageway may be completely sealed (e.g., a tube), partially open (e.g., a channel) or the passageway may define a region where the fluid stream is unrestricted by any tubing. The scanning area 142 may have other shapes and/or configurations in other embodiments. For example, the size of the tube (e.g., diameter, length, etc.) may vary in other embodiments. In another example, the scanning area 142 may be a tank (e.g., a transparent tank) through which the fluid stream may pass.

Data analysis device 148 includes a processor and a memory source. The processor is operable to execute program instructions. In one embodiment, the program instructions are operable to determine the presence and/or location of the compounds carried in the fluid stream as the fluid stream passes through the scanning area 142. In an embodiment, the data analysis device is capable of determining the concentration of the suspended material in the fluid stream as the fluid stream passes through the scanning area 142.

After the fluid stream passes through scanning area 142, the fluid stream is directed into appropriate flow channels and collected in one or more receiving containers. In one embodiment (not shown), a single receiving container is used to collect the fluid stream. A single container may be used when the data analysis device is used to identify the amount or size of the suspended material in the fluid stream. Under such analysis conditions, there may be no requirement to separate the fluid stream into individual containers.

In an embodiment, system 100 is used to separate the fluid stream into individual compounds. For example, if the fluid stream is composed of a mixture of two compounds, the system may further separate the compounds into individual containers. To allow separation of the compounds into individual storage containers, system 100 includes a stream splitter 150, a plurality of valves 160, and a plurality of receiving containers 170. Containers 110 and 170 may also be referred to as tanks.

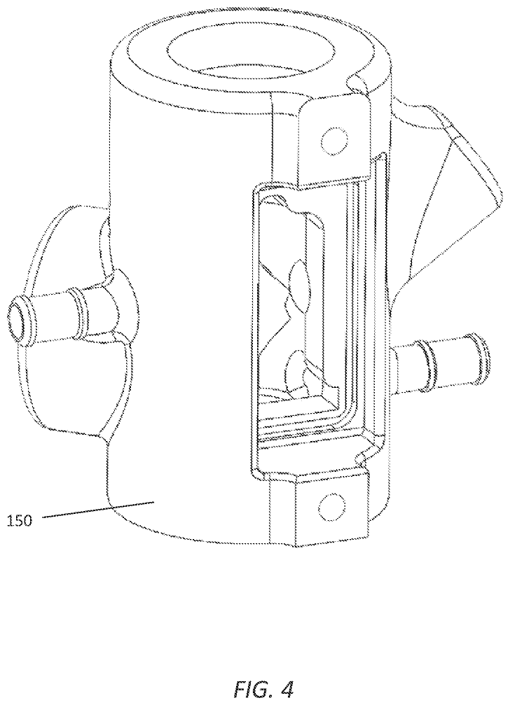

Stream splitter 150 receives the fluid stream from scanning area 142 and splits the stream into two or more passages. FIG. 4 shows a projection view of an exemplary splitter 150. In the embodiment depicted in FIG. 1, splitter 150 divides the flow stream into “n” passages, where n represents the number of receiving containers. Flow through each of the passages is controlled by valves 160a, 160b, . . . 160n. Each valve can be independently opened and closed to control the passage of the fluid stream through each of the passageways to receiving containers 170a, 170b, . . . 170n. In the depicted embodiment, the valves are shown as separate components. However, it should be understood that in some embodiments, the valves and splitter may be combined in a single unit.

Valves 160 may be any type of fluid control valve that is suitable for the fluid stream passing through the system. Valves 160 may be automatically controlled, for example, by coupling the valves to data analysis device 148. Alternatively, the valves may be manually controlled. The use of data analysis device 148 to operate the valves allows the system to automatically control the collection of the fluid streams into individual receiving containers 170.

In an automated system, data analysis device 148 automatically selects a receiving tank to collect the fluid passing through the detection system. Data analysis device 148 analyzes the fluid stream to determine the properties of compounds carried in the fluid stream. Based on the analysis of the compounds carried in the fluid stream, the data analysis device can operate the appropriate valves to direct the fluid stream into the appropriate receiving container 170. For example, to direct the fluid stream into receiving container 170a, all valves except valve 160a are closed. Similar control of the gate valves can be used to direct the fluid stream to the other receiving containers.

It should be understood that, while a single detection system is shown for the identification of compounds carried in a fluid stream, the system can be expanded by using multiple detection systems. Furthermore, multiple detection systems can be cascaded so that the detectors are arranged in series with one detector system feeding into another detector system. In this embodiment, the first detector in the series of detectors is used to determine if a portion of the fluid stream should be sent to the next downstream detector system or collected. In another embodiment, multiple detection systems may be in a parallel arrangement. In a parallel arrangement, multiple detection systems can be used to analyze a single incoming fluid stream split into multiple streams, or multiple incoming streams, substantially simultaneously. In another embodiment, an array of detection systems (detection systems arranged in both parallel and series) can be used for high throughput analysis of one or more fluid streams.

The criteria that are used to determine which receiving container is used to receive the fluid stream can be customized though through the data analysis device. In one embodiment, the concentration of compounds carried in the fluid stream may be used to determine which receiving container is used to collect the fluid stream. Each receiving container may be selected to receive a fluid stream having a predetermined range of concentrations of one or more of the compounds. For example, receiving container 170a may receive a fluid stream having a concentration of less than 50%, less than 45%, less than 40%, less than 35%, less than 30%, less than 25%, less than 20%, less than 15%, less than 10%, or less than 5% of a selected compound. Receiving container 170c may receive a fluid stream having more of a selected compound. For example, receiving container 170c may receive a fluid stream having a concentration of greater than 50%, greater than 55%, greater than 60%, greater than 65%, greater than 70%, greater than 75%, greater than 80%, greater than 85%, greater than 90%, or greater than 95% of a selected compound. Any fraction of the fluid stream that is not selected for receiving containers 170a or 170c can be directed to receiving container 170b.

While the above example describes the use of concentration for the selection of receiving containers, it should be understood that other criteria can be used to separate the fluid stream into different fractions. Examples of other criteria that can be used include, but are not limited to, identifying the chemical composition of the suspended compounds based on the interaction of the suspended compounds with the electromagnetic radiation from the electromagnetic radiation source, determining the shape of the compounds, determining the light absorbance spectrum of the compounds, and determining the size of the compounds. The system may also be capable of determining the presence of more than two different compounds of the fluid stream. For example, a fluid stream may include two different solid materials (e.g., small rock particles mixed with metal particles). The system may be configured to differentiate between two different types of solid particles and direct the particles to different collection containers.

After collection of the fractions of the fluid stream into separate containers, one or more of the containers may be used as a fluid stream source for further separation. For example, if container 170a is used to collect a fluid stream having less than 10% of the selected compound, and container 170c is used to collect a fluid stream having greater than 90% of the selected compound, then container 170b will have a mixed fraction that includes more than 10% of the selected compound, but less than 90% of the selected compound. In an embodiment, the mixed fraction from container 170b may be reintroduced into the system for further separation or may be sent to a different detection system for further processing. The mixed fraction from container 170b may, for example, be transferred to storage container 110 and used to create a new fluid stream for separation into fractions. Alternatively, a conduit (not shown) may be coupled between receiving container 170b and storage container 110 to allow direct transfer of the mixed fraction back into the system.

In an embodiment, a conduit 180 may be coupled between one or more of the receiving containers 170a-c and pump 120. For example, conduit 180 may be coupled to receiving container 170b, which collects a mixed fluid fraction. After the initial fluid sample is analyzed and transferred from the storage container 110 to the receiving containers, a mixed fraction (for example, in container 170b, may be reintroduced into the system through conduit 180. The reintroduction of the fluid from container 170b may be controlled by gate valve 185. This setup allows mixed fractions of the fluid stream to be recycled until substantially all of the initial fluid stream is separated into two substantially pure fractions. While this example describes the separation of a fluid stream based on two compounds in the fluid stream, it should be understood that the system could be expanded by adding more receiving containers to allow the separation of more than two compounds.

Example—Analysis and Separation of Hydrocarbons from a Water Stream

Hydrocarbon contamination of water sources is an ongoing problem around the world. Improper disposal of hydrocarbons, leaking hydrocarbon storage tanks, and accidents can lead to the hydrocarbons being released into water sources. Also, water is used in many petrochemical extraction operations, particularly hydraulic fracturing (“fracking”). Such uses generate large quantities of water having substantial hydrocarbon fractions. It is, therefore, desirable to have an efficient process for separating hydrocarbons from water.

A mixture of a liquid hydrocarbon in water is introduced into storage container 110 and allowed to settle such that the liquid hydrocarbon separates from the water into separate layers. Typically, the hydrocarbon layer will have a density that is less than the water layer, and the water layer will be at the bottom of the storage tank and the oil layer above the water layer. Removal of the fluids in the storage tank is performed through a conduit 115 disposed at the bottom of the storage tank (See FIG. 2).

Pump 120 is activated, drawing fluid from the bottom of storage container 110, creating a fluid stream that is sent to flow regulator 130. The fluid stream is then passed from the flow regulator to flow meter 135, where the flow rate is measured to ensure that the fluid stream is at the proper speed. The fluid stream is passed through scanning area 142, where the fluid stream is analyzed to determine the concentration of oil in the water stream.

As the oil is passed through scanning area 142, data analysis device 148 determines the concentration of oil droplets in the fluid stream. This may be done using the imaging and AI techniques described herein. The concentration of oil droplets in the fluid stream may be determined by measuring the number of droplets of oil in a known volume of water. Based on the concentration of oil droplets in the water, a determination may be made as to whether the fluid stream passing through the scanning area 142 is primarily an oil stream, a water stream, or a mixed stream. In the present embodiment, it can be expected that a water stream will initially pass through the scanning area 142, followed by a mixed water/oil stream, which is finally followed by a hydrocarbon stream. Each of these streams can then be directed into an individual receiving container 170a-c for collection.

Each receiving container 170a-c may be selected to receive water (170a), hydrocarbons (170b), or a mixed fluid (170c) having a predetermined concentration of suspended materials. For example, receiving container 170a may receive a fluid stream having a concentration of greater than 70%, greater than 75%, greater than 80%, greater than 85%, greater than 90%, or greater than 95% water. Receiving container 170c may receive a fluid stream having a concentration of greater than 70%, greater than 75%, greater than 80%, greater than 85%, greater than 90%, or greater than 95% of hydrocarbons. Any fraction of the fluid stream that is not selected for receiving containers 170a or 170c can be directed to receiving container 170b.

To improve the yield of separated water from oil, the mixed fluid stream is reintroduced into the system, and the steps of analysis and separation are repeated. This process can be repeated any number of times until substantially all of the initial oil-water mixture has been separated into individual water and oil containers.

### Example—Separation of Metal Containing Particles

A mixture of metal containing particles in water is introduced into storage container 110. The metal containing particles can be substantially pure metal particles, metal containing mineral particles, or a combination of metal particles and mineral particles. The mixture may also include dirt, sand or other materials that were associated with the collection of the metals or minerals. A mixer in the storage container is used to agitate the mixture of metal containing particles in water. The fluid carrying the metal containing particles in the storage tank is removed through a conduit 115 disposed at the bottom of the storage tank (See FIG. 2).

Pump 120 is activated, drawing fluid from the bottom of storage container 110, creating a fluid stream carrying the metal containing particles that is sent to flow regulator 130. The fluid stream is then passed from the flow regulator to flow meter 135, where the flow rate is measured to ensure that the fluid stream is at the proper speed. The fluid stream is passed through scanning area 142, where the fluid stream is analyzed to determine the properties of the metal containing particles.

As the fluid stream is passed through scanning area 142, data analysis device 148 determines the properties of the metal particles in the fluid stream. This may be done using the imaging and AI techniques described herein. In one test, the chemical identity of the particles is determined by measuring the light absorbance, light reflectance or light emission of the particles after light irradiation in the detection system. In other tests, the shape and/or size of the metal containing particles can be used to determine the identity of the particles. Based on the chemical identity of the particles, a determination is made as to whether the fluid stream passing through the scanning area 142 is a stream containing, primarily, a first type of metal containing particle, a second type of metal containing particle, or is a mixed stream of both types of metal containing particles. Each of these streams is directed into an individual receiving container 170a-c for collection.

Each receiving container 170a-c may be selected to receive the first type of metal containing particles (170a), the second type of metal containing particles (170c), or a mixed fluid (170b) having both the first type of metal containing particles and the second type of metal containing particles in a predetermined concentration range. For example, receiving container 170a may receive a fluid stream having a concentration of greater than 70%, greater than 75%, greater than 80%, greater than 85%, greater than 90%, or greater than 95% of the first type of metal containing particles. Receiving container 170c may receive a fluid stream having a concentration of greater than 70%, greater than 75%, greater than 80%, greater than 85%, greater than 90%, or greater than 95% of the second type of metal containing particles. Any fraction of the fluid stream that is not selected for receiving containers 170a or 170c can be directed to receiving container 170b.

To improve the yield of separated metal containing particles, the mixed fluid stream is reintroduced into the system, and the steps of analysis and separation are repeated. This process can be repeated any number of times until substantially all of the fluid stream has been separated into individual container based on the type of metal containing particle.

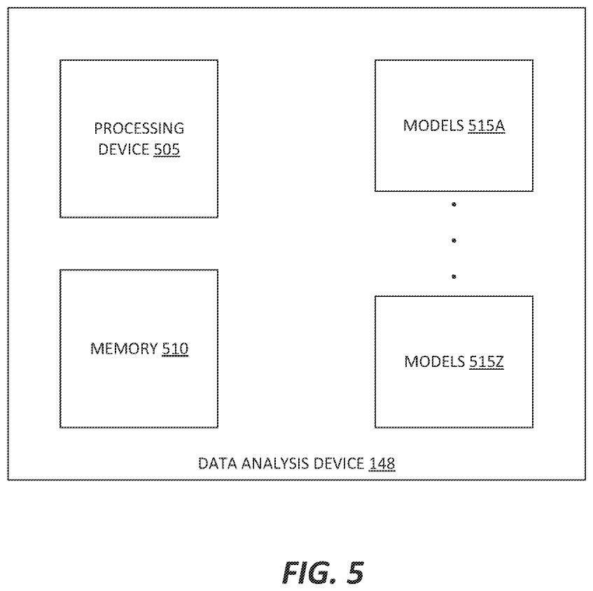

FIG. 5 is a block diagram illustrating an example data analysis device 148, in accordance with one or more embodiments of the present disclosure. The data analysis device 148 may be a computing device. The computing device may be connected to other computing devices in a network, LAN, an intranet, an extranet, and/or the Internet. The computing device may operate in the capacity of a server machine in a client-server network environment or in the capacity of a client in a peer-to-peer network environment. The computing device may be provided by a personal computer (PC), a server computer, a smartphone, a cellular phone, a laptop computer, a desktop computer, a tablet computer, or any machine capable of executing a set of instructions (sequential or otherwise) that specify actions to be taken by that machine. Further, while only the disclosure may refer to a single computing device, the term “computing device” shall also be taken to include any collection of computing devices that individually or jointly execute a set (or multiple sets) of instructions to perform the methods discussed herein.

The data analysis device 148 includes a processing device 505 (e.g., a general-purpose processor, a microprocessor, a programmable logic device (PLD), a processing core, etc.), a memory 510 (e.g., main memory, random access memory, hard disk drive (HDD) solid-state drive (SSD), flash memory, hybrid disk drive, etc.), and models 515A through 515Z (e.g., neural networks, machine learning models, etc.). The processing device 505 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 505 may also comprise one or more special-purpose processing devices such as an application specific integrated circuit (ASIC), a field programmable gate array (FPGA), a digital signal processor (DSP), network processor, or the like. The processing device 505 may be configured to execute the operations described herein, in accordance with one or more aspects of the present disclosure, for performing the operations and steps discussed herein.

The machine learning models 515A through 515Z and/or components for executing/running the machine learning models 515A through 515Z (e.g., additional software, modules, etc.) may be stored in the memory 510. Memory 510 may include a computer-readable storage medium (e.g., flash memory, a hard disk drive, or some other non-transitory computer/machine-readable medium) on which instructions for carrying out the operations and/or instructions for the machine learning models 515A through 515Z may be stored. The instructions may also reside, completely or at least partially, memory 510 and/or within processing device 505 during execution thereof by data analysis device 148.

Data analysis device 148 may further include other optional components, devices, systems, etc. For example, data analysis device 148 may include a network interface device that may communicate with the network (e.g., a wireless network, a cellular network, etc.). The data analysis device 148 also may include a video display unit (e.g., a liquid crystal display (LCD), a cathode ray tube (CRT), a touch screen, etc.), and an input device (e.g., a keyboard, a mouse, etc.) and an acoustic signal generation device (e.g., a speaker).

As illustrated in FIG. 5, the data analysis device 148 includes models 515A through 515Z. Each of the models 515A through 515Z may be used to detect different types of compounds carried in the fluid stream. For example, model 515A may be used to detect oil (e.g., the suspended material) in water. In another example, model 515B may be used to detect particles of lead (e.g., the suspended material) in water. In a further example, model 515C may be used to detect particles of dirt and particles of copper in water.

The models 515A through 515Z may detect, identify, etc., suspended materials based on different factors, parameters, criteria, etc., in different embodiments. In one embodiment, one or more of the models 515A through 515Z may determine a concentration of the suspended materials in the fluid. For example, model 515A may determine the concentration (in terms of parts per million (PPM)) of a type of particle in a fluid. In another embodiment, one or more of the models 515A through 515Z may identify a chemical composition of the suspended materials based on the interaction of the suspended materials with the light from the light source. For example, the composition of the suspended materials may be determined based on the brightness of the light that is detected after the light passes through the fluid and/or suspended materials. In a further embodiment, one or more of the models 515A through 515Z may determine a size and/or a shape of the suspended materials. For example, the model 515A may determine whether a particle is oval-shaped and/or is within a certain size range (e.g., 5 nanometers to 10 nanometers). In some embodiments, one or more of the models 515A through 515Z may determine the light absorption spectrum of the suspended material. For example, model 515A may determine the color of the light that is detected after the light passes through the fluid and/or suspended materials.

The models 515A through 515Z may be neural networks that have been trained to detect, identify, etc., different suspended materials within a fluid stream. The models 515A through 515Z may be trained using different sets of training data. For example, model 515A may be trained using images and/or video (e.g., training data) of oil mixed with water to allow model 515A to identify oil and water within a fluid stream (e.g., identify the differences in color, consistency, viscosity, etc., between oil and water). In another example, the models 515A may be trained using spectrograph data (e.g., data generated by a spectrograph, which may include images, waveforms, etc.). The data that is used to train the models 515A through 515Z may be based on the type of fluid and/or suspended materials that are used with the identification/separation system (e.g., system 100 illustrated in FIG. 1). For example, different images, videos, waveforms, tabular data, graphs, time-series data, etc., may be provided to different models, which may be used to detect different types of suspended materials in different types of fluids.

In some embodiments, the data analysis device 148 may allow for different models to be used. For example, different models may be installed on the data analysis device 148. In another example, an existing model on the data analysis device may be updated to a newer version of the model. This allows the data analysis device 148 to be more modular. For example, if a user wants to detect a different type of suspended particle (which may be in a different type of fluid), the user may install a new model that is trained to detect that type of suspended particle. This allows the data analysis device 148 to be coupled to different systems. For example, the same data analysis device 148 may be used with a first system for detecting/separating oil from water, and may be used with a second system for detecting/separating lead particles from water.

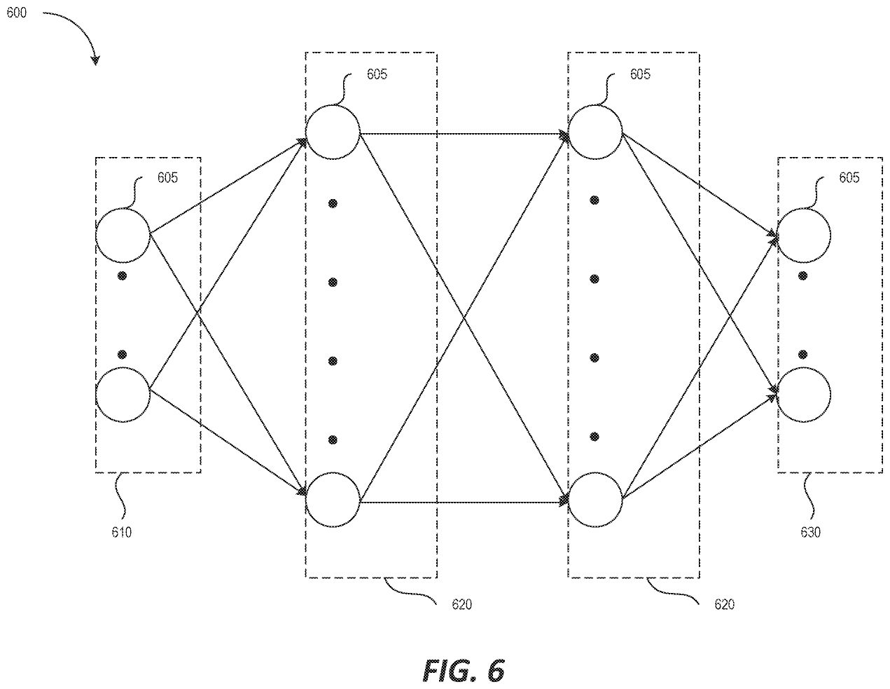

FIG. 6 is a diagram illustrating an example neural network 600, in accordance with one or more embodiments of the present disclosure. The neural network 600 may be an example of one type of model (e.g., one type of machine learning model) that may be used to identify and/or separate suspended materials from a fluid. The neural network 600 may be used to model relationships between (e.g., complex) inputs and outputs or to find patterns in data, where the dependency between the inputs and the outputs may not be easily ascertained. The neural network 600 may also be a computing model that may be used to determine a feature in input data through various computations. For example, the neural network 600 may determine a feature (e.g., a number, shape, pattern, etc.) in input data (e.g., audio data, image data, video data, etc.) according to a structure that defines a sequence of computations to be performed. The neural network 600 may be a convolutional neural network (CNN). A CNN may be a feed-forward neural network. A feed-forward neural network may be a type of neural network where the connections between the nodes do not form a cycle. For example, the signals, messages, data, information, etc., flow forward from the input layer 610 (e.g., from the input nodes), through intermediate layers 620, to the output layer 630 (e.g., to the output nodes) of the neural network 600 from left to right. The signals, messages, data, information, etc., may not go back through the neural network (e.g., may not go from right to left). A CNN may be used for image analysis. The connections and/or their associated weights may take the form of a convolutional filter (and/or a convolutional kernel) that may be applied to an input (e.g., may be applied to different pixels of an image). Although the present disclosure may refer to image analysis for CNNs, in other embodiments, the CNN may be used for other types of data and inputs.

The neural network 600 includes an input layer 610, intermediate layers 620, and an output layer 630. Each of the input layer 610, the intermediate layers 620, and the output layer 630 includes one or more nodes 605. Each of the input layer 610, the intermediate layers 620, and the output layer 630 may have a different number of nodes 605. The neural network 600 may be a deep neural network (DNN) or a deep CNN. A neural network may be deep (e.g., a deep neural network) if there is more than one intermediate layer 620 (e.g., if there are four, ten, or some other appropriate number of intermediate layers 620). As illustrated in FIG. 1, the neural network 600 includes two intermediate layers 620 (e.g., two columns of nodes 605). In one embodiment, an intermediate layer 620 may include nodes 605 and connections/weights that are coupled to the nodes 605 in the intermediate layer 620. The nodes of an intermediate layer may receive input for the intermediate layer 620 (e.g., an output, such as a feature map, generated by a previous layer). The weights (e.g., a kernel/filter) may be applied to the inputs to generate an output of the current intermediate layer (e.g., a feature map).

Each of the nodes 605 in a layer is connected to either a node 605 in the next level (e.g., next sub-layer) or a node 605 in another layer, as represented by the arrows/lines between the nodes 605. For example, the nodes 605 in the input layer are each coupled to at least one node 605 in the first intermediate layer 620. Neural network 600 may be a fully connected neural network. For example, each node 605 in each layer or level is connected to each node in the subsequent layer or level where there is a subsequent layer or level (e.g., nodes 605 in the output layer 630 are not connected to other nodes).

Each connection may be associated with a weight or weight value (e.g., may have a weight). A weight or weight value may define coefficients applied to the computations. For example, the weights or weight values may be scaling factors between two or more nodes 605. Each node 605 may represent a summation of its inputs, and the weight or weight value associated with a connection may represent a coefficient or a scaling factor multiplied to an output of a node 605 in that connection. The weights between the nodes 605 may be determined, calculated, generated, assigned, learned, etc., during a training process for the neural network. For example, backpropagation may be used to set the weights such that the neural network 600 produces expected output values given corresponding values in labeled training data. Thus, the weights of the intermediate layers 620 can be considered as an encoding of meaningful patterns in the data. The weights of the connections between the nodes 605 may be modified by additional training.

Although neural network 600 is depicted with a particular number of nodes 605 layers and connections, various neural network architectures/configurations may be used in other embodiments. For example, different fully connected neural networks and partially connected neural networks (e.g., where all nodes in adjacent layers are not connected) may be used. In addition, although the present disclosure may refer to convolutional neural networks, other types of neural networks and/or deep neural networks may be used in other embodiments. For example, different fully connected neural networks and partially connected neural networks (e.g., where all nodes in adjacent layers are not connected) may be used. In addition, other types of neural networks may be used in other embodiments. For example, recurrent neural networks (RNN), long short-term memory (LSTM) neural networks, etc., may be used in other embodiments.

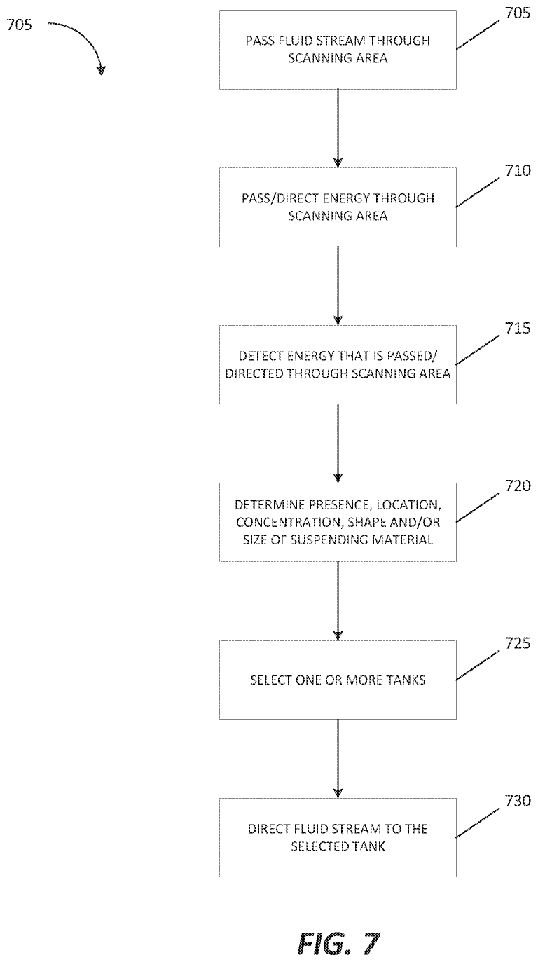

FIG. 7 is a flow diagram illustrating an example process for identifying/detecting suspended material and/or separating the suspended material from a fluid stream, in accordance with one or more embodiments of the present disclosure. Process 700 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the process 700 may be performed by one or more of a computing device, a detection/separation system, a data analysis device, etc.

With reference to FIG. 7, process 700 illustrates example functions used by various embodiments. Although specific function blocks (“blocks”) are disclosed in process 700, such blocks are examples. That is, embodiments are well suited to performing various other blocks or variations of the blocks recited in process 700. It is appreciated that the blocks in process 700 may be performed in an order different than presented, and that not all of the blocks in process 700 may be performed. In addition, additional other blocks (not illustrated in FIG. 7) may be inserted between the blocks illustrated in FIG. 7.

The process 700 begins at block 705, where the process 700 may pass a fluid stream through a scanning area. For example, the fluid may be pumped from a source (e.g., a source tank) through a scanning tube, through a tank (e.g., a scanning tank), etc. At block 710, a form of energy may be directed/passed through the scanning area. For example, electromagnetic radiation (e.g., radio waves, microwaves, infrared waves, visible light, ultraviolet rays, X-rays, gamma rays, etc.) may be directed/passed through the scanning area by an energy source (e.g., an emitter or transmitter). In another example, ultrasound waves may be directed/passed through the scanning area.

At block 715, the process 700 may detect the energy that is passed/directed through the scanning area as the fluid stream passes through the scanning area. For example, a camera, a light detector, an infrared detector, a receiver (e.g., a receiver for electromagnetic radiation), a detector, etc., may detect the energy that was directed/passed through the scanning area. At block 720, the process 700 may determine one or more of the presence, location, concentration, shape, and size of the suspended material within the fluid stream. For example, the process 700 may use data/information generated by the detector and a model (e.g., a machine learning model, a neural network, etc.) to determine the presence of the suspending material.

At block 725, the process 700 may select one or more tanks to receive the fluid stream based on one or more of the presence, location, concentration, shape, and size of the suspended material within the fluid stream. For example, if the presence of a suspended material is detected within the fluid stream (e.g., detected by the model), the process 700 may select a first tank. If the suspended material is not detected within the fluid stream, the process 700 may select a second tank. At block 730, the process 700 may direct the fluid stream to the selected tank. For example, if the first tank is selected, the process 700 may direct the fluid stream to the first tank, as discussed above.

The blocks 705 through 730 may be repeated in a loop until various conditions, parameters, criteria, etc., are met. For example, the blocks 705 through 730 may be repeated until the amount of suspended material within the fluid reaches a threshold amount. In another example, the blocks 705 through 730 may be repeated until the concentration of suspended material within the fluid reaches a threshold concentration. In a further example, blocks 705 through 730 may be repeated as long as oil is detected in a fluid stream that includes oil and water, as discussed above.

Although the present disclosure may refer to machine learning models, such as a neural network, other types of machine learning and/or artificial intelligence systems, algorithms, etc., may be used to detect and/or identify suspended materials. For example, support vector machines, supervised learning, semi-supervised learning, unsupervised learning, regression analysis, boosting, Bayesian networks, etc., may be used in other embodiments.

Unless specifically stated otherwise, terms such as “controlling,” “determining,” “providing,” “generating,” “indicating,” obtaining,” “coupling,” “receiving,” “causing,” “training,” “updating,” or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein, are meant as labels to distinguish among different elements and may not necessarily have an ordinal meaning according to their numerical designation.

Examples described herein also relate to an apparatus for performing the operations described herein. This apparatus may be specially constructed for the required purposes, or it may comprise a general-purpose computing device selectively programmed by a computer program stored in the computing device. Such a computer program may be stored in a computer-readable non-transitory storage medium.

The methods and illustrative examples described herein are not inherently related to any particular computer or other apparatus. Various general-purpose systems may be used in accordance with the teachings described herein, or it may prove convenient to construct more specialized apparatus to perform the required method steps. The required structure for a variety of these systems will appear as set forth in the description above.

The above description is intended to be illustrative and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

It should also be noted that in some alternative implementations, the functions/acts noted may occur out of the order noted in the figures. For example, two figures shown in succession may, in fact, be executed substantially concurrently, or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method of identifying and separating compounds carried in a fluid stream, the method comprising:
passing the fluid stream through a detection system;
determining the properties of compounds in the fluid stream as the fluid stream passes through the detection system;
selecting a flow channel for the fluid stream to be sent based on one or more properties of the compounds in the fluid stream;
directing the fluid stream to the selected flow channel to separate the compounds.

2. The method of claim 1, wherein the fluid stream is a liquid fluid stream, and wherein the liquid fluid stream comprises an immiscible fluid suspended in a liquid fluid.

3. The method of claim 1, wherein the fluid stream is a liquid fluid stream, and wherein the liquid fluid stream comprises one or more solid compounds carried in a liquid fluid.

4. The method of claim 1, wherein the fluid stream is a gaseous fluid stream, and wherein the gaseous fluid stream comprises one or more solid compounds carried in a gaseous fluid.

5. The method of claim 1, wherein determining the properties of the compounds comprises transmitting electromagnetic radiation into the fluid stream passing through the detection system and detecting a signal response from the electromagnetic radiation.

6-8. (canceled)

9. The method of claim 5, wherein determining the properties of the compounds comprises identifying the chemical composition of the compounds based on the interaction of the compounds with the electromagnetic radiation, and wherein selecting a flow channel for the fluid stream to be sent after leaving the detection system is based on the chemical composition of the compounds in the fluid stream.

10. The method of claim 1, further comprising determining the concentration of the compounds in the fluid stream as the fluid stream passes through the detection system, wherein selecting a flow channel for the fluid stream to be sent after leaving the detection system is based on the concentration of the compounds in the fluid stream.

11. The method of claim 1, wherein determining the properties of the compounds further comprises determining the size of the compounds in the fluid stream as the fluid stream passes through the detection system, wherein selecting a flow channel for the fluid stream to be sent after leaving the detection system is based on the size of the compounds in the fluid stream.

12: The method of claim 1, wherein determining the properties of the compounds further comprises determining the shape of the compounds in the fluid stream as the fluid stream passes through the detection system, wherein selecting a flow channel for the fluid stream to be sent after leaving the detection system is based on the shape of the compounds in the fluid stream.

13. The method of claim 1, wherein determining the properties of the compounds is based on one or more machine learning models.

14. The method of claim 1, further comprising selecting one or more machine learning models based on one or more of the fluid and the compounds.

15. A system for identifying and separating compounds carried in a fluid stream, the system comprising:
a detection system configured to receive the fluid stream, wherein the detection system is configured to determine the properties of the compounds in the fluid stream as the fluid stream passes through the detection system;
a data analysis device coupled to the detector, wherein the data analysis device comprises a processor and a memory source, the processor operable to execute program instructions, and wherein the program instructions are operable to determine the properties of the compounds in the fluid stream as the fluid stream passes through the detection system;
one or more valves coupled to the detection system and the data analysis device; and
a plurality of flow channels coupled to the one or more valves;
wherein, during use, the data analysis device selects a flow channel for the fluid stream to be sent after leaving the detection system based on one or more properties of the compounds in the fluid stream; and wherein the data analysis device operates the one or more valves to direct the fluid stream to the selected flow channel after the fluid stream passes through the detection system to separate the material.

16-18. (canceled)

19. The system of claim 15, wherein the detection system comprises an electromagnetic radiation transmitter and an electromagnetic radiation detector, wherein the electromagnetic radiation transmitter and the electromagnetic radiation detector are positioned such that electromagnetic radiation from the electromagnetic radiation transmitter passes into the fluid stream passing through the detection system and the electromagnetic radiation passes from the fluid stream to the electromagnetic radiation detector.

20-22. (canceled)

23. The system of claim 19, wherein the detection system comprises a detection conduit through which the fluid stream passes through the detection system, wherein the detection conduit is composed of a material that is transparent to the electromagnetic radiation.

24. The system of claim 15, further comprising a storage container coupled to the detection system, wherein the storage container holds a mixture of the compounds in a fluid.

25. (canceled)

26. The system of claim 24, further comprising a pump coupled to the storage container and the detection system, wherein the pump receives the mixture of compounds in the fluid from the storage container and creates the fluid stream.

27. The system of claim 15, further comprising one or more receiving containers for receiving the fluid stream after the fluid stream passes through the one or more flow channels.

28. The system of claim 15, wherein the data analysis device further comprises one or more machine learning models to determine the properties of the compounds in the fluid stream.

29. The system of claim 15, the program instructions are further operable to: select the one or more machine learning models based on one or more of the fluid and the suspended materials.

30. A method of identifying and separating metal containing particles carried in a fluid stream, the method comprising:
passing the fluid stream through a detection system;
identifying properties of the metal containing particles in the fluid stream as the fluid stream passes through the detection system;
selecting a flow channel for the fluid stream to be sent after leaving the detection system based on the properties of the metal containing particles in the fluid stream;
directing the fluid stream to the selected flow channel after the fluid stream passes through the detection system to separate the metal containing particles.

31-35. (canceled)
